(19) United States
(12) Patent Application Publication
(10) Pub. No.: US 2024/0359032 A1
(43) Pub. Date: Oct. 31, 2024

(54) RETINAL PROSTHESIS AND VISUAL PERCEPTION METHOD BASED ON RETINAL PROSTHESIS

(71) Applicant: Westlake University, Hangzhou (CN)

(72) Inventors: Chuanqing WANG, Hangzhou (CN); Jie YANG, Hangzhou (CN); MOHAMAD SAWAN, Hangzhou (CN)

(21) Appl. No.: 18/648,286

(22) Filed: Apr. 26, 2024

(30) Foreign Application Priority Data
Apr. 28, 2023 (CN) 202310503566.5

(51) Int. Cl. A61N 5/06

(52) U.S. Cl. CPC A61N 5/0622; A61N 2005/0626; A61N 2005/0648

(57) ABSTRACT

Embodiments of the present disclosure relate to the biomedical technical field, and disclose a retinal prosthesis and a visual perception method based on the retinal prosthesis. The retinal prosthesis includes: a capturing assembly, a neuromorphic processor and a light stimulator. The capturing assembly is configured to capture an external scenario and encode the captured external scenario as spike sequences. The neuromorphic processor is configured to predict spike responses of ganglion cells of an implant recipient of the retinal prosthesis according to a preset deep learning algorithm and the spike sequences. The light stimulator is configured to stimulate the ganglion cells based on the spike responses of the ganglion cells. The retinal prosthesis can further reduce the data size and the amount of computation effectively, so that the power consumption is greatly reduced on the premise of keeping a relatively high processing speed.

### CROSS REFERENCE TO RELATED APPLICATIONS

The present application claims the benefit of priority under the Paris Convention to Chinese Patent Application No. 202310503566.5 filed on Apr. 28, 2023, which is incorporated herein by reference in its entirety.

### TECHNICAL FIELD

Embodiments of the present disclosure relate to the biomedical technical field, and particularly relate to a retinal prosthesis and a visual perception method based on the retinal prosthesis.

### BACKGROUND

Optogenetics-based retinal prosthesis are used to treat two visual diseases: age-related macular degeneration (AMD) and retinitis pigmentosa (RP). More than 100 million people worldwide suffer from these two types of visual degenerative diseases. As shown in FIG. 1, AMD and RP patients partially loss visual perception due to the degeneration of a large amount of cone and rod cells in the retina. The differences between the two diseases are the location of the degenerated cone and rod cells, as well as the order of cell apoptosis. At present, existing drug and gene therapy methods have limited effects on severe AMD and RP patients. With the development of optogenetics, retinal prosthesis based on optogenetics have become an important treatment for these two diseases.

The cone and rod cells of a normal functional retina can effectively perceive external scenes and generate electroneurographic signals to encode these external scenes. Bipolar cells and other cell layers process corresponding electroneurographic signals and then transmit the processed neural code to the cerebral cortex. A retinal prosthesis uses a deep learning model to replace the signal processing capability of a normal retina to perceive and process scenes. Then it stimulates the ganglion cells with light stimulation method to fire similar neural spike with normal retina. The ganglion cells are located in the outermost layer of the retina. After these generated neural spikes are transmitted to the visual cortex, they form partial visual perception.

However, inventors of the present disclosure have found that most retinal prostheses in the industry capture an image of external scenario with a head-mounted camera, and project the image to the retina by means of near infrared laser after simple image processing. An array of photodiodes implanted beneath the retina can convert the near infrared laser into a stimulating current, so as to generate a corresponding visual perception. However, such retinal prosthesis only supports simple image processing with a quite limited visual recovery effect, and it is hard to realize a benign balance between the processing speed and the power consumption.

### SUMMARY

Embodiments of the present disclosure are intended to provide a retinal prosthesis and a visual perception method based on the retinal prosthesis. The retinal prosthesis can further reduce the data size and the amount of computation effectively, so that the power consumption is greatly reduced on the premise of keeping a relatively high processing speed. To solve the above technical problems, the embodiments of the present disclosure provide a retinal prosthesis, including: a capturing assembly, a neuromorphic processor and a light stimulator. The capturing assembly is configured to capture an external scenario and encode the captured external scenario as spike sequences. The neuromorphic processor is configured to predict spike responses of ganglion cells of an implant recipient of the retinal prosthesis according to a preset deep learning algorithm and the spike sequences. The light stimulator is configured to stimulate the ganglion cells of the implant recipient of the retinal prosthesis based on the spike responses of the ganglion cells, allowing the implant recipient to gain a visual perception.

The embodiments of the present disclosure further provide a visual perception method based on a retinal prosthesis, which is adapted to the retinal prosthesis and includes capturing an external scenario and encoding the captured external scenario as spike sequences, predicting spike responses of ganglion cells according to a preset deep learning algorithm and the spike sequences; and stimulating the ganglion cells of an implant recipient of the retinal prosthesis based on the spike responses of the ganglion cells, allowing the implant recipient to gain a visual perception.

According to the retinal prosthesis and the visual perception method based on the retinal prosthesis provided in the embodiments of the present disclosure, the retinal prosthesis includes the capturing assembly, the neuromorphic processor, and the light stimulator sequentially connected. The capturing assembly is configured to capture the external scenario and encode the captured external scenario as the spike sequences. The neuromorphic processor is configured to predict the spike responses of the ganglion cells according to the preset deep learning algorithm and the spike sequences. The light stimulator is configured to stimulate the ganglion cells of the implant recipient of the retinal prosthesis based on the spike responses of the ganglion cells, allowing the implant recipient of the retinal prosthesis to gain a visual perception. The processing algorithm of the existing retinal prosthesis in the industry is quite simple, and only simple image processing is performed, so the visual recovery effect is limited. Moreover, realizing the benign balance between processing speed and power consumption is urgent and necessary. In contrast, the retinal prosthesis provided in the embodiments of the present disclosure performs bionic full-spike processing, and procedures of external scenario acquiring and signal processing are both performed in the form of spike so that the power consumption is extremely low, and the data size and the amount of computation are effectively decreased. Hence, the proposed solution can improve the restoration effect and reduce power consumption, which is a promising way to bring convenience to blind people with retinal prostheses.

In some embodiments, the neuromorphic processor includes a spiking recurrent model, and is specifically configured to implement the spiking recurrent model to obtain, by inputting the spike sequences into the spiking recurrent model, the spike responses of the ganglion cells predicted by the spiking recurrent model. Using the spiking recurrent model to predict the spike responses of the ganglion cells avoids the use of floating-point multiplication, further reducing the data size and the amount of computation

In some embodiments, while the neuromorphic processor is running the spiking recurrent model, data computation in the spiking recurrent model is performed by way of concurrent computation and by virtue of the sparsity of the spike sequences.

In some embodiments, the spiking recurrent model includes a plurality of layers, and the neuromorphic processor performing the data computation in the spiking recurrent model by way of concurrent computation specifically includes: for two sequentially connected layers of the spiking recurrent model, the neuromorphic processor performing data storage and reading in a first mode at (2n−1)^{th} time step of a first layer, and the neuromorphic processor performing data storage and reading in a second mode at (2n)^{th} time step of the first layer. Read-only memories responsible for storing and reading corresponding to the first mode and the second mode are different, and the n is an integer greater than 0. In view of the fact that the read-only memories cannot be read in and out at the same time, the neuromorphic processor has to wait in a case that the two sequentially connected layers share one read-only memory for data storage and reading. In contrast, embodiments of the present disclosure provide two modes, the data between the two layers is stored and read at the (2n−1)^{th} time step of the first layer in the first mode, and is stored and read at the (2n)^{th} time step of the first layer in the second mode. The read-only memories responsible for storing and reading are different in the two modes. Such configuration may warrant concurrent computation, thereby greatly improving the computing, running and processing speeds of the spiking recurrent model.

In some embodiments, in the first mode, the neuromorphic processor stores a calculation result at a current time step of the first layer into a first read-only memory and controls a second layer to read data from a second read-only memory. In the second mode, the neuromorphic processor stores the calculation result at the current time step of the first layer into the second read-only memory and controls the second layer to read data from the first read-only memory. The overall neuromorphic processor runs the spiking recurrent model by using a ping-pong data storage and reading structure, further improving the signal processing capacity.

In some embodiments, the plurality of layers include a plurality of spike layers and a plurality of recurrent layers, each of the plurality of spike layers including a weight static random access memory (SRAM), a spike buffer, a control unit, a plurality of neurons and a membrane potential calculation unit. The weight SRAM is configured to store a weight value for a current spike layer. The spike buffer is configured to store inputted spike sequences. The inputted spike sequences are externally inputted spike sequences or outputted spikes of a previous spike layer. The control unit is configured to send the weight value and the inputted spike sequences into the membrane potential calculation unit. The membrane potential calculation unit is configured to calculate a membrane potential change value of each of the plurality of neurons according to the weight value and the inputted spike sequences, and to send the membrane potential change value to a corresponding one of the plurality of neurons. The plurality of neurons are configured to output spikes. Outputted spikes of the current spike layer are stored in the spike buffer of a subsequent layer.

In some embodiments, the membrane potential calculation unit includes an array of processing elements. The array of processing elements includes a plurality of processing elements, the time step is divided into a plurality of time periods according to a preset division criterion, and the inputted spike sequences are divided into a plurality of sub-spike sequences according to a number of the time periods. The control unit is specifically configured to first assign the weight value to each of the plurality of processing elements and then respectively send the plurality of sub-spike sequences into target input processing elements in the array of processing elements according to an order of the time periods. Each of the plurality of sub-spike sequences flows diagonally in the array of processing elements until it is outputted by a target output processing element corresponding to a respective one of the target input processing elements. The array of processing elements performing multistage concurrent computation accelerates the computation greatly, achieving real-time processing of the inputted spike sequences and thereby improving the speed of visual perception performed by the retinal prosthesis.

In some embodiments, the membrane potential calculation unit is further configured to not store the calculation result of one of the plurality of processing elements in a case that it is determined that the weight value assigned to the processing element is equal to 0 and/or a sub-spike sequence corresponding to the processing element is equal to 0. In view of the fact that most energy in the calculation process is consumed in a storage procedure, embodiments of the present disclosure skip meaningless storage by virtue of the fine-grained sparsity of the spike sequences, effectively reducing the power consumption.

In some embodiments, the control unit is further configured to skip a calculation for the current spike layer based on a convolution kernel to directly transfer the inputted spike sequences to a subsequent layer in a case that the weight value is equal to 0. Embodiments of the present disclosure skip the unnecessary calculation based on the convolution kernel by virtue of the coarse-grained sparsity of spike sequences, so that the data size and the amount of computation are effectively reduced, and the processing speed and power consumption of the retinal prosthesis are further balanced, thereby better meeting requirements of patients.

In some embodiments, the capturing assembly includes an event camera and a recording apparatus. The event camera is configured to capture the external scenario. The recording apparatus is configured to encode the external scenario captured by the event camera as spike sequences.

In some embodiments, the light stimulator includes a data converter and a stimulation apparatus. The data converter is configured to convert the spike responses of the ganglion cells into light stimulation signals. The stimulation apparatus is configured to stimulate the ganglion cells of the implant recipient of the retinal prosthesis through the light stimulation signals, allowing the implant recipient to gain a visual perception.

### BRIEF DESCRIPTION OF THE DRAWINGS

One or more embodiments are illustrated through the diagrams in the corresponding drawings. These exemplary descriptions do not constitute a limitation on the embodiments.

FIG. 1 is a schematic diagram of visual degeneration in AMD patients and RP patients;

FIG. 2 is a schematic diagram of a retinal prosthesis provided in an embodiment of the present disclosure;

FIG. 3 is a schematic diagram of a capturing assembly provided in an embodiment of the present disclosure;

FIG. 4 is a schematic diagram of a model architecture of a spiking recurrent model provided in an embodiment of the present disclosure;

FIG. 5 is a schematic diagram of a model architecture of another spiking recurrent model provided in an embodiment of the present disclosure;

FIG. 6 is a schematic diagram of a light stimulator provided in an embodiment of the present disclosure;

FIG. 7 is a schematic diagram showing data storage and reading in a first mode and data storage and reading in a second mode provided in an embodiment of the present disclosure;

FIG. 8 is a schematic diagram of a spike layer provided in an embodiment of the present disclosure;

FIG. 9 is a schematic diagram of an array of processing elements provided in an embodiment of the present disclosure;

FIG. 10 is a schematic diagram of a judging logic by virtue of the fine-grained sparsity of spike sequences provided in an embodiment of the present disclosure;

FIG. 11 is a flowchart of a visual perception method based on the retinal prosthesis provided in another embodiment of the present disclosure;

FIG. 12 is a diagram of comparison in spike sequence and spike count between recorded values of action potential responses of a normal retinal ganglion neuron to a dynamic video and predicted values of the spiking recurrent model provided in another embodiment of the present disclosure; and

FIG. 13 is a comparison diagram of validation results of the retinal prosthesis combined with retinal ganglion cells of a mouse provided in another embodiment of the present disclosure.

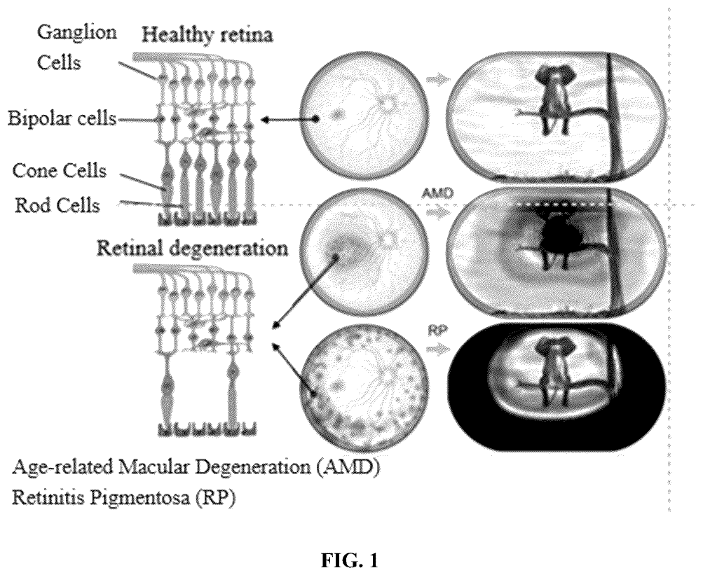

### DETAILED DESCRIPTION OF THE EMBODIMENTS

In order to make the objectives, technical solutions and advantages of embodiments of the present disclosure clearer, the embodiments of the present disclosure will be described in detail below in conjunction with the accompanying drawings. However, those of ordinary skill in the art may understand that in the embodiments of the present disclosure, many technical details are provided to enable readers to better understand the present disclosure. However, even without these technical details and various variations and modifications based on the following embodiments, the technical solutions as claimed in the present disclosure may also be achieved. The division of the following embodiments is for the convenience of description and should not constitute any limitation on the specific implementation of the present disclosure. The embodiments may be combined with and referenced to each other without contradiction.

Some embodiments of the present disclosure relates to a retinal prosthesis. The implementation details of the retinal prosthesis in this embodiment are specified below. The following content is only for the convenience of understanding the provided implementation details and is not essential for implementing this solution.

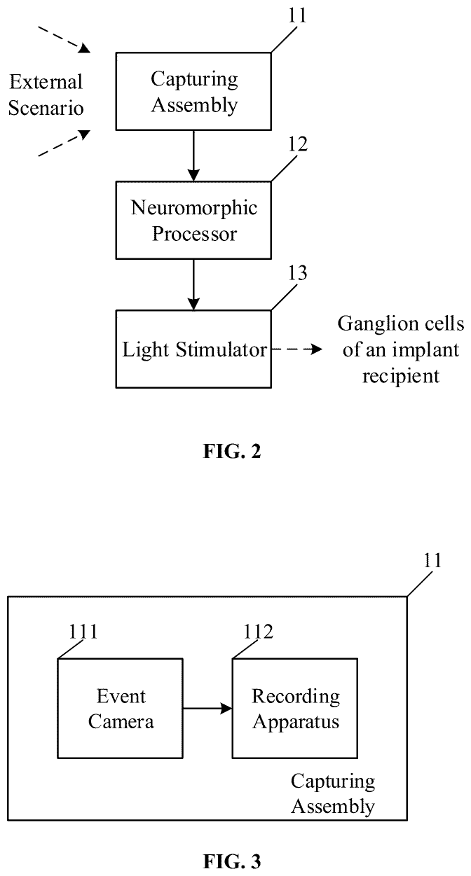

The retinal prosthesis in this embodiment may, as shown in FIG. 2, include: a capturing assembly 11, a neuromorphic processor 12 and a light stimulator 13. The neuromorphic processor 12 is respectively connected to the capturing assembly 11 and the light stimulator 13.

The neuromorphic processor 12 may be integrated in a printed circuit board (PCB).

The capturing assembly 11 is configured to capture an external scenario, encode the captured external scenario as spike sequences, and transmit the encoded spike sequences into the neuromorphic processor 12.

In some embodiments, the capturing assembly 11 may, as shown in FIG. 3, include an event camera 111 and a recording apparatus 112. The event camera 111 is configured to capture the external scenario, and the recording apparatus 112 is configured to encode the external scenario captured by the event camera 111 as spike sequences. The event camera 111 may be a dynamic vision sensor (DVS), an asynchronous time-based image sensor (ATIS), a dynamic and active pixel vision sensor (DAVIS), or the like.

The neuromorphic processor 12 is configured to predict spike responses of ganglion cells according to a preset deep learning algorithm and the spike sequences transmitted in by the capturing assembly 11. The preset deep learning algorithm may be selected and set by those skilled in the art according to actual requirements. For example, a convolutional neural network may be used.

In some embodiments, the neuromorphic processor includes a spiking recurrent model, and is specifically configured to run the spiking recurrent model to obtain, by inputting the spike sequences transmitted in by the capturing assembly 11 into the spiking recurrent model, the spike responses of the ganglion cells predicted by the spiking recurrent model.

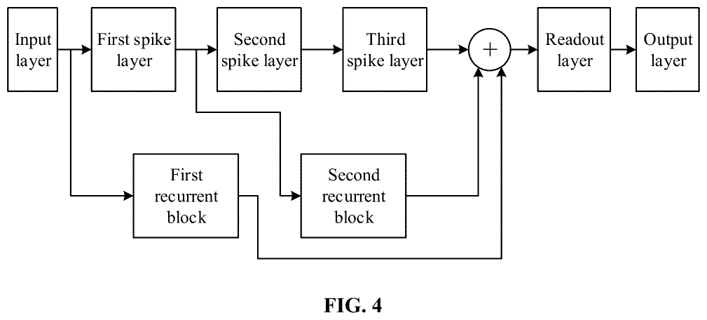

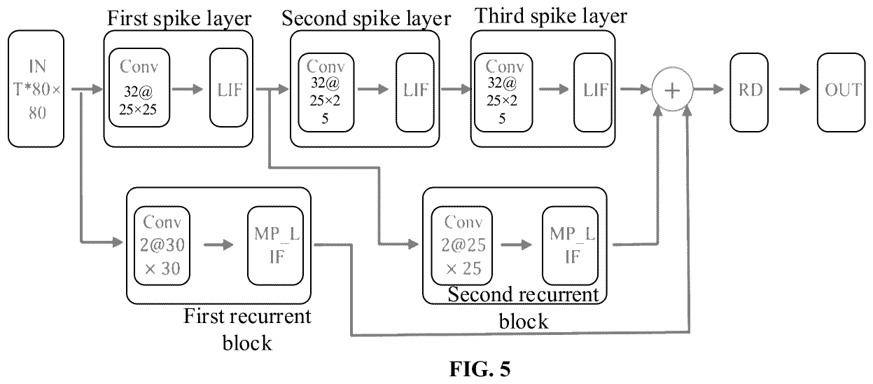

In some embodiments, the spiking recurrent model may be as shown in FIG. 4 and FIG. 5. The model architecture of the spiking recurrent model includes an input layer, a first spike layer, a second spike layer, a third spike layer, a first recurrent layer, a second recurrent layer, a readout layer and an output layer. The first spike layer, the second spike layer and the third spike layer are sequentially connected. The input layer is connected with the first spike layer and the first recurrent layer respectively. The first spike layer is also connected with the second recurrent layer. The readout layer is connected with the third spike layer, the first recurrent layer and the second recurrent layer respectively (the third spike layer, the first recurrent layer and the second recurrent layer may be connected with the readout layer through an adder). The output layer is connected with the readout layer. As shown in FIG. 4, each spike layer includes a convolutional layer and leaky integrate and fire (LIF) neurons, and each recurrent layer includes a convolutional layer and membrane potential LIF (MP_LIF) neurons. The number of LIF neurons in the first spike layer is greater than the number of LIF neurons in the second spike layer, and the number of LIF neurons in the second spike layer is greater than the number of LIF neurons in the third spike layer. The number of MP_LIF neurons in the first recurrent layer is equal to the number of MP_LIF neurons in the second recurrent layer and the number of LIF neurons in the third spike layer.

In some embodiments, while the neuromorphic processor 12 is running the spiking recurrent model, data computation in the spiking recurrent model is performed by way of concurrent computation and by virtue of the sparsity of the spike sequences.

The light stimulator 13 is configured to stimulate the ganglion cells of an implant recipient of the retinal prosthesis based on the spike responses of the ganglion cells, allowing the implant recipient to gain a visual perception.

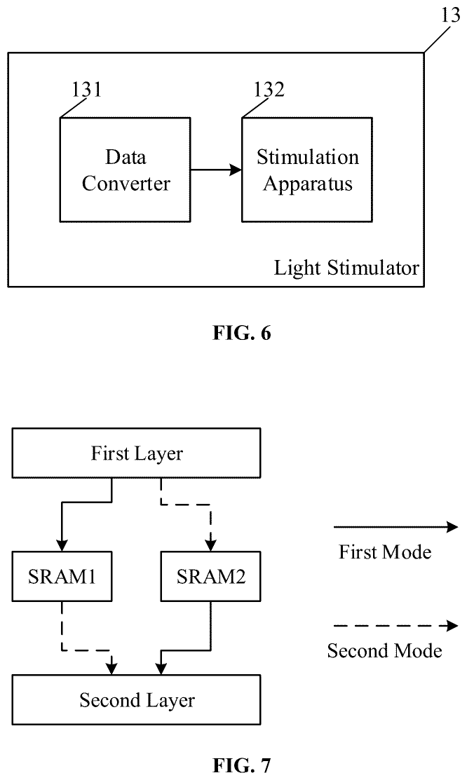

In some embodiments, the light stimulator 13 may, as shown in FIG. 6, include a data converter 131 and a stimulation apparatus 132. The data converter 131 is configured to convert the spike responses of the ganglion cells into light stimulation signals, and the stimulation apparatus 132 is configured to stimulate the ganglion cells of the implant recipient of the retinal prosthesis through the light stimulation signals, allowing the implant recipient to gain a visual perception.

In the embodiments, the retinal prosthesis includes the capturing assembly, the neuromorphic processor and the light stimulator sequentially connected. The capturing assembly is configured to capture the external scenario and encode the captured external scenario as the spike sequences. The neuromorphic processor is configured to predict the spike responses of the ganglion cells based on the preset deep learning algorithm and the spike sequences. The light stimulator is configured to stimulate the ganglion cells of the implant recipient of the retinal prosthesis based on the spike responses of the ganglion cells, allowing the implant recipient of the retinal prosthesis to gain a visual perception. The processing procedure of the manure retinal prosthesis in the industry is quite simple, and only simple image processing is performed therein, so the visual recovery effect is quite limited. Moreover, it is hard to realize the benign balance between the processing speed and the power consumption. In contrast, the retinal prosthesis provided in the embodiments of the present disclosure performs bionic full-spike processing, and procedures of external scenario acquiring and signal processing are both performed in the form of spike, so that the power consumption thereof is extremely low, and the data size and the amount of computation are effectively reduced. That is, the power consumption is greatly reduced on the premise of keeping the relatively high processing speed, the visual perception ability of the retinal prosthesis is also improved, and the eyesight of the implant recipient is recovered as far as possible.

In some embodiments, the spiking recurrent model includes a plurality of layers, and the neuromorphic processor performing the data computation in the spiking recurrent model by way of concurrent computation specifically includes: for two sequentially connected layers of the spiking recurrent model, the neuromorphic processor performing data storage and reading in a first mode at (2n−1)^{th} time step of a first layer of the two sequentially connected layers, and the neuromorphic processor performing data storage and reading in a second mode at (2n)^{th} time step of the first layer of the two sequentially connected layers. Read-only memories responsible for storing and reading corresponding to the first mode and the second mode are different, and the n is an integer greater than 0.

Specifically speaking, the static random access memory, also known as static read-only memory, cannot write and read at the same time. If two sequentially connected layers share one read-only memory for data storage and reading, the neuromorphic processor will have to wait, wasting a lot of time. In contrast, this embodiment provides two modes, the neuromorphic processor performs data storage and reading between two layers at the (2n−1)^{th} time step of the first layer in the first mode, and at the (2n)^{th} time step of the first layer in the second mode. The read-only memories responsible for storing and reading are different in the two modes. Such configuration may warrant concurrent computation, thereby greatly improving the computing, running and processing speeds of the spiking recurrent model.

In some embodiments, data storage and reading in the first mode and data storage and reading in the second mode may be as shown in FIG. 7. In the first mode, the neuromorphic processor stores a calculation result at a current time step of the first layer into a first read-only memory (SRAM1) and controls the second layer to read data from a second read-only memory (SRAM2). In the second mode, the neuromorphic processor stores the calculation result at the current time step of the first layer into the second read-only memory (SRAM2) and controls the second layer to read data from the first read-only memory (SRAM1). The overall neuromorphic processor runs the spiking recurrent model by using a ping-pong data storage and reading structure, further improving the signal processing capacity.

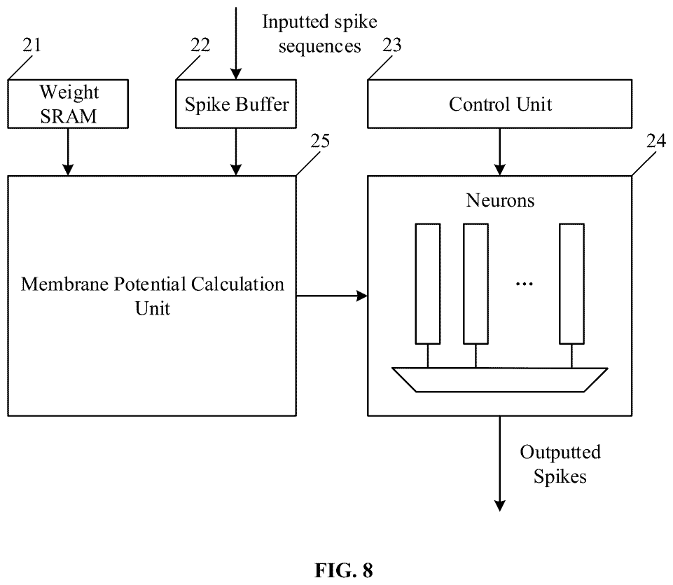

In some embodiments, the plurality of layers include a plurality of spike layers and a plurality of recurrent layers, each of the plurality of spike layers as shown in FIG. 8 may include a weight SRAM 21, a spike buffer 22, a control unit 23, a plurality of neurons 24 and a membrane potential calculation unit 25.

The weight SRAM 21 is configured to store a weight value for a current spike layer.

The spike buffer 22 is configured to store the inputted spike sequences. The inputted spike sequences are externally inputted spike sequences or outputted spikes of a previous spike layer.

The control unit 23 is configured to send the weight value stored in the weight SRAM 21 and the inputted spike sequences stored in the spike buffer 22 into the membrane potential calculation unit 25.

The membrane potential calculation unit 25 is configured to calculate a membrane potential change value of each of the plurality of neurons 24 according to the weight value and the inputted spike sequences, and to send the membrane potential change value to a corresponding one of the plurality of neurons 24.

The plurality of neurons 24 are configured to output spikes. Outputted spikes of the current spike layer are stored in the spike buffer 24 of a subsequent layer.

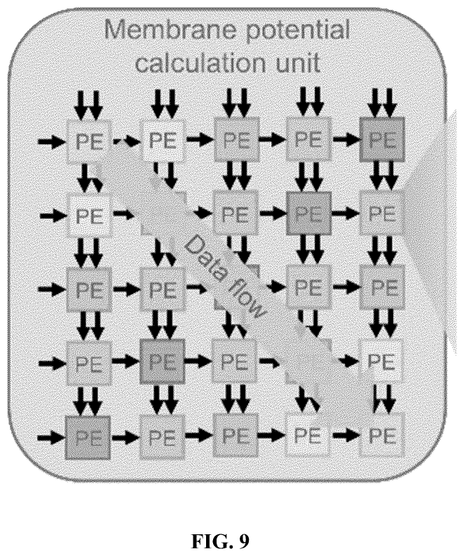

In some embodiments, the membrane potential calculation unit 25 includes an array of processing elements. The array of processing elements includes a plurality of processing elements, as shown in FIG. 9. The time step of the current spike layer is divided into a plurality of time periods according to a preset division criterion, and the inputted spike sequences stored in the spike buffer 22 are divided into a plurality of sub-spike sequences according to a number of the time periods. The control unit 23 is specifically configured to first assign the weight value to each of the plurality of processing elements in the array of processing elements and then respectively send the plurality of sub-spike sequences into target input processing elements in the array of processing elements according to an order of the time periods. Each of the plurality of sub-spike sequences flows diagonally in the array of processing elements until it is outputted by a target output processing element corresponding to a respective one of the target input processing elements.

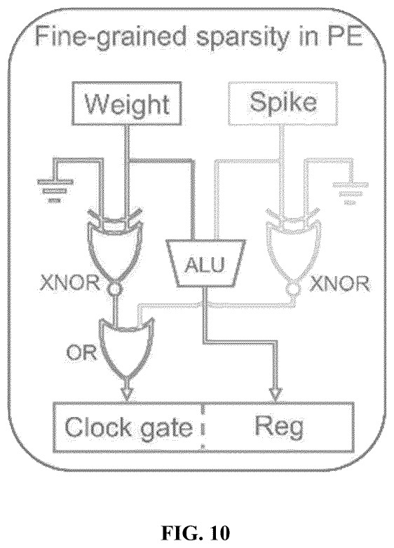

In some embodiments, the membrane potential calculation unit 25 is further configured to not store the calculation result of one of the plurality of processing elements in a case that it is determined that the weight value assigned to the processing element is equal to 0 and/or a sub-spike sequence corresponding to the processing element is equal to 0. In view of the fact that most energy in the calculation process is consumed in a storage procedure, this embodiment skips meaningless storage by virtue of the fine-grained sparsity of the spike sequences, effectively reducing the power consumption. FIG. 10 shows a judging logic by virtue of the fine-grained sparsity of spike sequences.

In some embodiments, the control unit 23 is further configured to skip a calculation for the current spike layer based on a convolution kernel to directly transfer the inputted spike sequences to the subsequent layer in a case that the weight value is equal to 0. That is, this embodiment skips the unnecessary calculation based on the convolution kernel by virtue of the coarse-grained sparsity of spike sequences, so that the data size and the amount of computation are effectively reduced, and the processing speed and power consumption of the retinal prosthesis are further balanced, thereby better meeting requirements of the implant recipient.

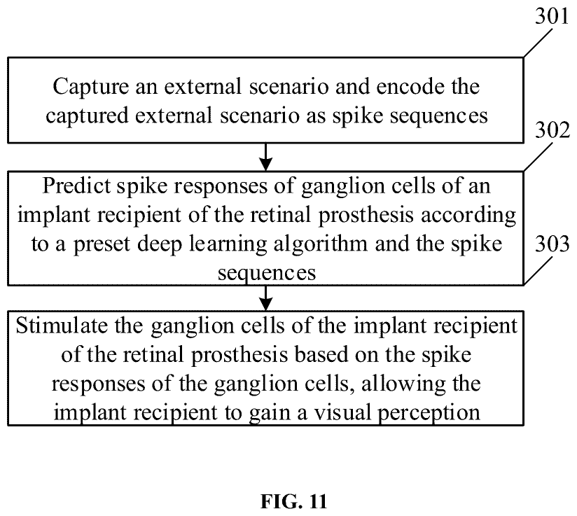

Some embodiments of the present disclosure relate to a visual perception method based on a retinal prosthesis, which is adapted to the retinal prosthesis. Implementation details of the visual perception method based on the retinal prosthesis in this embodiment are specified below. The following content is only for the convenience of understanding the provided implementation details and is not essential for implementing this solution. The visual perception method based on a retinal prosthesis in this embodiment may be as shown in FIG. 11, specifically including:

301, capturing an external scenario and encoding the captured external scenario as spike sequences;

302, predicting spike responses of ganglion cells according to a preset deep learning algorithm and the spike sequences; and

303, stimulating the ganglion cells of an implant recipient of the retinal prosthesis based on the spike responses of the ganglion cells, allowing the implant recipient to gain a visual perception.

It is worth noting that the modules involved in this embodiment all are logical modules. In an actual application, one logical model may be a physical unit or a part of a physical unit or may be a combination of a plurality of physical units. In addition, to highlight the innovated part of the present disclosure, units not closely related to the technical problem proposed in the present disclosure are not introduced in this embodiment, which, by no means, indicates that there are no other units existing in this embodiment.

The operation division of the aforementioned methods is only for the purpose of clear description. When implemented, combination into one operation, or splitting of some operation into more operations, as long as the same logical relationship is included, are within the scope of protection of the present disclosure; and adding irrelevant modifications or introducing irrelevant designs to an algorithm or a process, but not changing the core design of the algorithm and the process, is within the scope of protection of the present disclosure.

In some embodiments, the performance qualification results of the neuromorphic processor (a neuromorphic chip) are introduced.

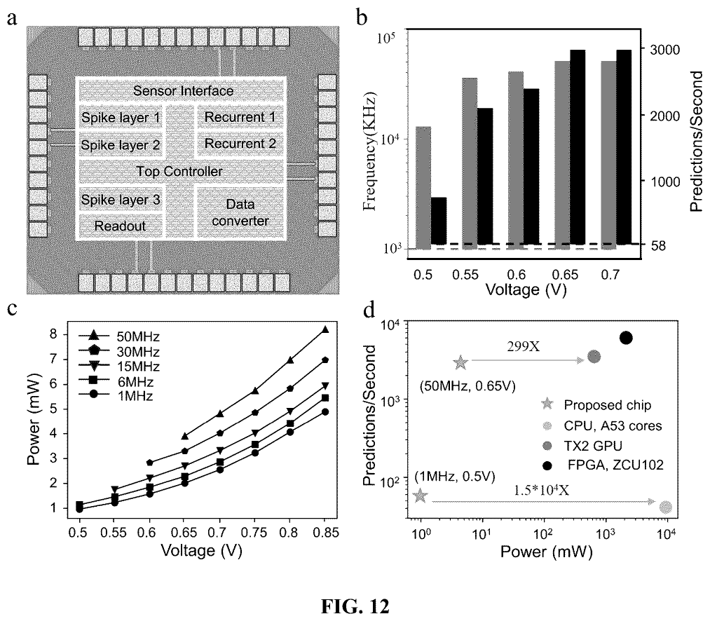

The neuromorphic chip may be manufactured in a TSMC 40 nm CMOS process and packaged in a QFN-64 package. The area of the chip is 1.12 square millimeters, and its layout details are shown in (a) of FIG. 12. A controller at the top is responsible for controlling data streams among several processing modules such as the spike layers and the recurrent layers. A sensor interface module undertakes a data pre-processing task.

Performance of the chip is measured through experiments. The major concern focuses on delay and power. As shown in (b) of FIG. 12, a working range of the chip varies according to an input voltage. This is because a low input voltage cannot support the chip to work at a high clock frequency. Therefore, at the input voltage of 0.5 V, the clock frequency range of the chip is limited to a very short range. As the voltage rises, the working frequency range increases as well. In addition, the computation speed of the chip is highly dependent on the clock frequency. A high throughput of this chip can support 2980 predictions per second at an input voltage of 0.65V. A low throughput thereof can support 58 predictions per second. Due to the fact that the spiking recurrent model predicts the number of spikes fired by the ganglion cell within 33 milliseconds, the throughput of this chip is required to complete 30 predictions per second. The chip can still meet the real-time processing requirements of the retinal prosthesis at an input voltage of 0.45V.

(c) of FIG. 12 illustrates the power of the chip at different voltages and clock frequencies. When it works at 1 MHz and 0.45V, the minimum power is 585 ρW. This is our proposed optimal working point. The inputted spikes can be processed in real time with minimal energy consumption. Finally, we compared this neuromorphic chip with commercial CPUs, GPUs, and FPGAs, as shown in (d) of FIG. 12. From the perspective of embedded devices, we have chosen A53 core, TX2 GPU, and ZCU102 as representative devices. When the chip operates at 1 MHz and 0.5V, its processing speed is slightly better than A53 core, but the power is greatly reduced. Compared to TX2 GPU and ZCU102, this chip can support a similar processing speed under a low power condition. In summary, this low-power real-time processing neuromorphic chip can better serve the retinal prosthesis.

Some embodiments introduce the biological validation results of the retinal prosthesis.

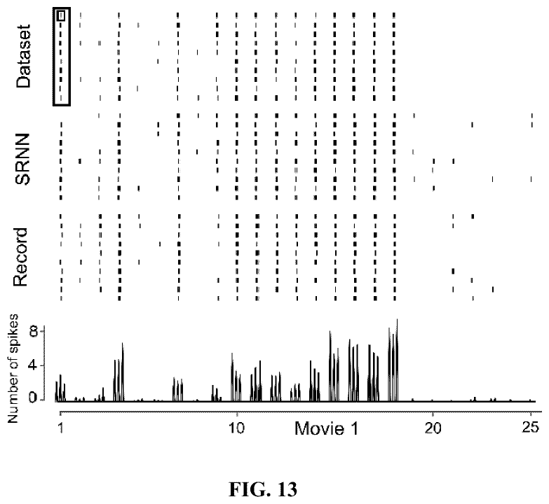

The retinal prosthesis in the above embodiment is implanted into a mouse, and its processing and stimulation effects on the retinal ganglion cells of the mouse are validated. As shown in FIG. 13, Dataset represents the action potentials collected from a mouse with normal vision, while SRNN represents the predicted action potentials based on the retinal prosthesis in the above embodiment. It can be found from the comparison of the action potentials that the retinal prosthesis has a good processing effect and can well fit the responses of the ganglion cells with normal function. Record represents action potential signals collected by the retinal prosthesis stimulating the ganglion cells cultured in vitro. It can be found from the comparison of the action potential signals that the retinal prosthesis has a good stimulating effect and is highly similar to a normal retina in response.

Those of ordinary skill in the art may understand that the aforementioned embodiments are specific embodiments for implementing the present disclosure, but in practical applications, various changes may be made in form and details without deviating from the spirit and scope of the present disclosure.

What is claimed is:

1. A retinal prosthesis, comprising: a capturing assembly, a neuromorphic processor and a light stimulator; wherein
the capturing assembly is configured to capture an external scenario and encode the captured external scenario as spike sequences;
the neuromorphic processor is configured to predict spike responses of ganglion cells of an implant recipient of the retinal prosthesis according to a preset deep learning algorithm and the spike sequences; and
the light stimulator is configured to stimulate the ganglion cells of the implant recipient of the retinal prosthesis based on the spike responses of the ganglion cells, allowing the implant recipient to gain a visual perception.

2. The retinal prosthesis according to claim 1, wherein the neuromorphic processor includes a spiking recurrent model, and is configured to run the spiking recurrent model to obtain, by inputting the spike sequences into the spiking recurrent model, the spike responses of the ganglion cells predicted by the spiking recurrent model.

3. The retinal prosthesis according to claim 2, wherein the neuromorphic processor is configured to run the spiking recurrent model by performing data computation in the spiking recurrent model by way of concurrent computation and by virtue of sparsity of the spike sequences.

4. The retinal prosthesis according to claim 3, wherein the spiking recurrent model includes a plurality of layers, and the neuromorphic processor performing the data computation in the spiking recurrent model by way of concurrent computation comprises:
for two sequentially connected layers of the spiking recurrent model, the neuromorphic processor performing data storage and reading in a first mode at (2n−1)^{th} time step of a first layer of the two sequentially connected layers, and the neuromorphic processor performing data storage and reading in a second mode at (2n)^{th} time step of the first layer of the two sequentially connected layers, wherein read-only memories responsible for storing and reading corresponding to the first mode and the second mode are different, and the n is an integer greater than 0.

5. The retinal prosthesis according to claim 4, wherein the neuromorphic processor is configured to
in the first mode, store a calculation result at a current time step of the first layer of the two sequentially connected layers into a first read-only memory and control a second layer of the two sequentially connected layers to read data from a second read-only memory; and
in the second mode, store the calculation result at the current time step of the first layer of the two sequentially connected layers into the second read-only memory and control the second layer of the two sequentially connected layers to read data from the first read-only memory.

6. The retinal prosthesis according to claim 4, wherein the plurality of layers include a plurality of spike layers and a plurality of recurrent layers, each of the plurality of spike layers comprising a weight static random access memory (SRAM), a spike buffer, a control unit, a plurality of neurons and a membrane potential calculation unit; wherein
the weight SRAM is configured to store a weight value for a current spike layer;
the spike buffer is configured to store inputted spike sequences, wherein the inputted spike sequences are externally inputted spike sequences or outputted spikes of a previous spike layer;
the control unit is configured to send the weight value and the inputted spike sequences into the membrane potential calculation unit;
the membrane potential calculation unit is configured to calculate a membrane potential change value of each of the plurality of neurons according to the weight value and the inputted spike sequences, and to send the membrane potential change value to a corresponding one of the plurality of neurons; and
the plurality of neurons are configured to output spikes, wherein outputted spikes of the current spike layer are stored in the spike buffer of a subsequent layer.

7. The retinal prosthesis according to claim 6, wherein
the membrane potential calculation unit comprises an array of processing elements, the array of processing elements comprises a plurality of processing elements, the time step is divided into a plurality of time periods according to a preset division criterion, and the inputted spike sequences are divided into a plurality of sub-spike sequences according to a number of the time periods; and
the control unit is further configured to first assign the weight value to each of the plurality of processing elements and then respectively send the plurality of sub-spike sequences into target input processing elements in the array of processing elements according to an order of the time periods; wherein each of the plurality of sub-spike sequences flows diagonally in the array of processing elements until it is outputted by a target output processing element corresponding to a respective one of the target input processing elements.

8. The retinal prosthesis according to claim 7, wherein the membrane potential calculation unit is further configured to not store the calculation result of one of the plurality of processing elements in a case that it is determined that the weight value assigned to the processing element is equal to 0 and/or a sub-spike sequence corresponding to the processing element is equal to 0.

9. The retinal prosthesis according to claim 8, wherein the control unit is further configured to skip a calculation for the current spike layer based on a convolution kernel to directly transfer the inputted spike sequences to the subsequent layer in a case that the weight value is equal to 0.

10. The retinal prosthesis according to claim 1, wherein the capturing assembly includes an event camera and a recording apparatus, wherein
the event camera is configured to capture the external scenario; and
the recording apparatus is configured to encode the external scenario captured by the event camera as spike sequences.

11. The retinal prosthesis according to claim 1, wherein the light stimulator includes a data converter and a stimulation apparatus, wherein
the data converter is configured to convert the spike responses of the ganglion cells into light stimulation signals; and
the stimulation apparatus is configured to stimulate the ganglion cells of the implant recipient of the retinal prosthesis through the light stimulation signals, allowing the implant recipient to gain a visual perception.

12. A visual perception method based on a retinal prosthesis, adapted to the retinal prosthesis according to claim 1 and comprising:
capturing an external scenario and encoding the captured external scenario as spike sequences;
predicting spike responses of ganglion cells according to a preset deep learning algorithm and the spike sequences; and
stimulating the ganglion cells of an implant recipient of the retinal prosthesis based on the spike responses of the ganglion cells, allowing the implant recipient to gain a visual perception.
